SEQUENCE LISTING (2 sequences)

SEQ ID NO: 1 - DNA, 20 bp, synthetic construct
  source 1..20: mol_type = other DNA
agagtttgatcctggctcag

SEQ ID NO: 2 - DNA, 20 bp, synthetic construct
  source 1..20: mol_type = other DNA
ctacggctaccttgttacga

(19) United States
(12) Patent Application Publication
(10) Pub. No.: US 2026/0273000 A1
(43) Pub. Date: Sep. 17, 2026

(54) PROBIOTIC WITH EFFICACY IN IMPROVING VAGINITIS, COMPOUND MICROECOLOGICAL PREPARATION, AND APPLICATION THEREOF

(71) Applicant: Ningbo University, Ningbo (CN)

(72) Inventors: Xiaoqun ZENG, Ningbo (CN); Yan TENG, Ningbo (CN); Daodong PAN, Ningbo (CN); Ling LI, Ningbo (CN); Zhen WU, Ningbo (CN); Qiwei DU, Ningbo (CN); Ruonan YAN, Ningbo (CN); Tao ZHANG, Ningbo (CN)

(21) Appl. No.: 19/671,393

(22) Filed: May 8, 2026

(30) Foreign Application Priority Data
Jun. 16, 2025 (CN) 202510799017.6

(51) Int. Cl. A61K 35/747; A61P 15/02; A61P 31/04; A61P 31/10

(52) U.S. Cl. CPC A61K 35/747; A61P 15/02; A61P 31/04; A61P 31/10

(57) ABSTRACT

The present disclosure discloses a probiotic with efficacy in improving vaginitis, a compound microecological preparation, and an application thereof, the probiotic is characterized by comprising at least one of the following strains: Lactiplantibacillus plantarum 22-9 strain with a deposit number CGMCC No. 33867, Lactiplantibacillus plantarum XA-9 strain with a deposit number CGMCC No. 33868, and Lactiplantibacillus pentosus strain B-1-35 with a deposit number CGMCC No. 33870, the probiotic compound microecological preparation is prepared by inoculating a lactic acid bacteria starter culture, which is obtained by mixing Lactiplantibacillus plantarum 22-9, Lactiplantibacillus plantarum XA-9, and Lactiplantibacillus pentosus strain B-1-35 cultures in a volume ratio of 2:1:3, into a fermentation medium at an inoculation volume of 1-5%, the preparation has the advantages of increasing the content of lactic acid bacteria in the vagina, reducing the content of pathogenic bacteria.

### SEQUENCE LISTING

The Sequence Listing XML file named “Sequence_Listing.xml,” created on Jun. 5, 2025, and having a size of 2,746 bytes, contains the sequence listing for this application and is hereby incorporated by reference in its entirety.

### TECHNICAL FIELD

The present disclosure relates to a probiotic compound microecological preparation, in particular to a probiotic with efficacy in improving vaginitis, a compound microecological preparation, and an application thereof.

### BACKGROUND

Vaginitis is characterized by symptoms such as vulvovaginal itching, burning sensation, irritation, pain, a “fishy” vaginal odor, and abnormal vaginal discharge. Vaginitis can affect reproductive system health, increase the risk of contracting sexually transmitted infections, induce other gynecological inflammations, impact fertility, and elevate the risks of miscarriage, preterm birth, infertility, and ectopic pregnancy. The affected population accounts for approximately 26% to 33% of all women, with a recurrence rate of about 72% for vaginitis. The recurrence rate of Vulvovaginal Candidiasis (VVC) is as high as 89%. Bacterial vaginosis (BV), which constitutes the largest proportion of vaginitis cases, and VVC are primarily caused by Gardnerella vaginalis and Candida albicans. After adhering and colonizing the vaginal environment, these pathogens aggregate with other pathogenic bacteria to form biofilms, resisting the host's immune defense mechanisms and exacerbating the symptoms of vaginitis.

When the vagina is heavily invaded by pathogens, the body can regulate itself through vaginal self-regulatory mechanisms. These include: The secretion of acidic discharges to expel some pathogens; the recognition and phagocytosis of pathogens by innate immune cells in the vaginal environment to initiate an immune response and prevent the spread of infection; and the action of lactic acid bacteria in the vagina, which produce substances such as lactic acid and hydrogen peroxide to create and maintain an acidic environment, thereby inhibiting the growth of other harmful bacteria. However, the body's self-regulatory capacity is limited and cannot eliminate pathogens from the vagina, necessitating therapeutic intervention. Currently, treatments for vaginal infections are primarily divided into pharmacotherapy and biotherapy. While pharmacotherapy inhibits pathogenic bacteria, it also adversely affects the vaginal lactic acid bacterial flora, often leading to recurrence and chronic infections. In contrast, biotherapeutic approaches, such as treatment with lactic acid bacteria, inhibit the invasion and overgrowth of exogenous and opportunistic pathogens, offering distinct advantages such as low recurrence rates, high safety, minimal side effects, and no issues with drug resistance. For the health of the vaginal microecosystem, there is an urgent need to develop a probiotic compound microecological preparation with efficacy in improving vaginitis-related symptoms.

### SUMMARY

A technical scheme to be addressed by the present disclosure is to provide a probiotic with efficacy in improving vaginitis, a compound microecological preparation, and an application thereof, which may increase the content of lactic acid bacteria in the vagina, reduce the content of pathogenic bacteria, reduce the number of inflammatory cells in the vagina, and reduce levels of inflammatory factors interleukin-1β (IL-1β), interleukin-6 (IL-6) and tumor necrosis factor-α (TNF-α) in serum.

A technical scheme adopted by the present disclosure to solve the above technical problems is:

The present disclosure provides a probiotic with efficacy in improving vaginitis, the probiotics include at least one of Lactiplantibacillus plantarum 22-9 strain with a deposit number CGMCC No. 33867 at the China General Microbiological Culture Collection Center, Lactiplantibacillus plantarum XA-9 strain with a deposit number CGMCC No. 33868, and Lactiplantibacillus pentosus B-1-35 strain with a deposit number CGMCC No. 33870.

The present disclosure also provides a probiotic compound microecological preparation with efficacy in improving vaginitis, the probiotic includes at least one of Lactiplantibacillus plantarum 22-9 strain with a deposit number CGMCC No. 33867, Lactiplantibacillus plantarum XA-9 strain with a deposit number CGMCC No. 33868, and Lactiplantibacillus pentosus B-1-35 strain with a deposit number CGMCC No. 33870, where the active ingredient of the probiotic compound microecological preparation includes the probiotic bacteria.

In some embodiments, the number of viable bacteria in the probiotic compound microecological preparation is not less than 1×10⁷ CFU/mL.

In some embodiments, a concentration of Lactiplantibacillus plantarum 22-9 bacteria solution, Lactiplantibacillus plantarum XA-9 bacteria solution and Lactiplantibacillus pentosus B-1-35 bacteria solution is the same and the mixed volume ratio is 2:1:3.

The present disclosure also provides a method for preparing the probiotic compound microecological preparation with efficacy in improving vaginitis, including the following steps:
(1) Inoculating Lactiplantibacillus plantarum 22-9, Lactiplantibacillus plantarum XA-9, and Lactiplantibacillus pentosus B-1-35 into a fermentation medium at a volume ratio of 1-5% (v/v), and culturing at 35-40° C. for 15-20 h to obtain a lactic acid bacterial culture, mixing the lactic acid bacterial cultures of Lactiplantibacillus plantarum 22-9, Lactiplantibacillus plantarum XA-9, and Lactiplantibacillus pentosus B-1-35 at a volume ratio of (1-3):(1-3):(1-3) to obtain a mixed starter culture, inoculating mixed starter culture into the fermentation medium at a volume ratio of 2%, and culturing at 37° C. for 18 h to obtain a mixed culture of lactic acid bacteria;
(2) centrifuging the lactic acid bacterial culture obtained in step (1) to collect lactic acid bacteria pellet, and adjusting a cell concentration to 1×10⁷−1×10¹⁰ CFU/mL using Phosphate Buffered Saline (PBS), adding lyoprotectant of the same volume as sterile PBS, mixing and placing in a freezer at −80° C. overnight for pre-freezing, freeze-drying the pre-frozen sample to obtain a probiotic compound microecological preparation.

In some embodiments, in step (1), the lactic acid bacterial cultures of Lactiplantibacillus plantarum 22-9, Lactiplantibacillus plantarum XA-9, and Lactiplantibacillus pentosus B-1-35 are mixed in a volume ratio of 2:1:3 to obtain a mixed starter culture, and the bacterial concentration of each lactic acid bacterial culture is the same.

In some embodiments, the fermentation medium described in step (1) is prepared by dissolving an MRS solid medium with 1 L of distilled water and sterilizing the MRS solid medium at 121° C. for 15 min, the formula of the MRS solid medium is 10 g of peptone, 10 g of beef extract, 5 g of yeast extract, 2 g of diammonium citrate, 5 g of sodium acetate, 20 g of glucose, 1 mL of Tween 80, 0.5 g of magnesium sulfate, 0.25 g of manganese sulfate, and 15 g of agar powder.

In some embodiments, the formula of the lyoprotectant described in step (2) is 118.2 g/L of trehalose, 17.1 g/L of L-cysteine, 10.3 g/L of sorbitol, 1.7 g/L of sodium acetate, and 120 g/L of skim milk powder.

The present disclosure also provides an application of the above-mentioned probiotics in the preparation of Gardnerella vaginalis and/or Candida albicans inhibitors.

The present disclosure also provides an application of the probiotic compound microecological preparation for the treatment of vaginitis symptom improvement or therapeutic drugs.

Compared with the existing technologies, advantages of this present disclosure are as follows: A probiotic with efficacy in improving vaginitis-related symptoms, a compound microecological preparation, and an application thereof, three strains of lactic acid bacterial with significant inhibitory effects on Gardnerella vaginalis and Candida albicans are selected, compatibility of the microecological preparations obtained by using three kinds of lactic acid bacteria is scientific and reasonable, which may greatly inhibit the growth of vaginal pathogenic bacteria, effectively increase the proportion of lactic acid bacteria in the vagina, reduce the number of inflammatory cells in the vagina, reduce levels of inflammatory factors IL-1β, IL-6, and TNF-α in serum, alleviate excessive inflammatory reaction, and have obvious improvement effect on mice with vaginitis model, it makes up for a gap of probiotics products on the market to alleviate symptoms of vaginitis.

The Lactiplantibacillus plantarum 22-9 strain, with the deposit number CGMCC No. 33867, is deposited at the CGMCC on Mar. 19, 2025, and the preservation address is No. 3, No. 1 West Beichen Road, Chaoyang District, Beijing.

The Lactiplantibacillus plantarum XA-9 strain, with the deposit number CGMCC No. 33868, is deposited at the CGMCC on Mar. 19, 2025, and the preservation address is No. 3, No. 1 West Beichen Road, Chaoyang District, Beijing.

The Lactiplantibacillus pentosus B-1-35 strain, with the deposit number CGMCC No. 33870, is deposited at the CGMCC on Mar. 19, 2025, and the preservation address is No. 3, No. 1 West Beichen Road, Chaoyang District, Beijing.

### BRIEF DESCRIPTION OF THE DRAWINGS

FIG. 1 is the inhibition rate of lactic acid bacteria against Gardnerella vaginalis;

FIG. 2 is the inhibition rate of lactic acid bacteria against Candida albicans;

FIG. 3 is the growth and acid production curve of lactic acid bacteria;

FIGS. 4A-4B are the survival rates and Optical Density (OD) values of lactic acid bacteria at different pH levels, where FIG. 4A is the OD value of lactic acid bacteria at different pH levels, FIG. 4B is the survival rate of lactic acid bacteria at pH 3.0; FIG. 4C is the survival rate of lactic acid bacteria at pH 3.5;

FIGS. 5A-5D are the survival rates and the OD values of lactic acid bacteria under different bile salts, where FIG. 5A is the OD value of lactic acid bacteria under different bile salts, FIG. 5B is the survival rate of lactic acid bacteria at a bile salt concentration of 0.3%; FIG. 5C is the survival rate of lactic acid bacteria at a bile salt concentration of 0.6%; FIG. 5D is the survival rate of lactic acid bacteria at a bile salt concentration of 0.9%;

FIGS. 6A-6B are the evaluations of the adhesion capability of lactic acid bacteria: FIG. 6A is the auto-aggregation capability of lactic acid bacteria; FIG. 6B is the hydrophobicity of lactic acid bacteria;

FIG. 7 is the hemolysis experiment of three strains of lactic acid bacteria;

FIG. 8 is the transmission electron microscopy images of the three strains of lactic acid bacteria at 30,000× magnification;

FIG. 9 is the electrophoresis of 16S rDNA PCR products of three strains of lactic acid bacteria;

FIG. 10 is the phylogenetic tree of three strains of lactic acid bacteria;

FIG. 11 is the inhibitory capability of the microecological preparation against Gardnerella vaginalis at different compounding ratios of lactic acid bacteria;

FIG. 12 is the inhibitory capability of the microecological preparation against Candida albicans at different compounding ratios of lactic acid bacteria;

FIG. 13 is the aerobic plate count of the undiluted microecological preparation before and after freeze-drying;

FIG. 14 is the aerobic plate count of the undiluted microecological preparation at different storage temperatures;

FIG. 15 is the Gram staining of the vaginal lavage fluid on day 4 of the administration period in the mouse model of vaginitis;

FIG. 16 is the vaginal and uterine tissues after the administration period in the mouse model of vaginitis;

FIG. 17 is the results of Hematoxylin and Eosin staining (HE) staining of mouse vaginal tissues at 400× magnification (inflammatory cells are indicated by black arrows);

FIG. 18 is the results of Periodic Acid-Schiff staining (PAS) staining of mouse vaginal tissues at 200× magnification (fungal positivity is indicated by green arrows);

FIGS. 19A-19B are the number of vaginal inflammatory cells and squamous epithelial cells of mouse vaginal tissues, where FIG. 19A is the number of vaginal inflammatory cells, FIG. 19B is the number of squamous epithelial cells; the letters a-c in the FIG. 19: different letters indicate significant differences between different treatment groups (P<0.05);

FIGS. 20A-20C is the levels of interleukin-1β (IL-1β), interleukin-6 (IL-6), and tumor necrosis factor-α (TNF-α) in the serum of mice in each group, where FIG. 20A: IL-1β; where FIG. 20B: IL-6; where FIG. 20C: TNF-α; the letters a-c in the FIG. 20: different letters indicate significant differences between different treatment groups (P<0.05);

FIG. 21 is the effect of the probiotic compound microecological preparation on the relative abundance of species at the phylum level of intestinal bacteria;

FIG. 22 is the effect of the probiotic compound microecological preparation on the relative abundance of species at the genus level of intestinal bacteria;

FIG. 23 is the effect of the probiotic compound microecological preparation on the relative abundance of species at the phylum level of vaginal bacteria;

FIG. 24 is the effect of the probiotic compound microecological preparation on the relative abundance of species at the genus level of vaginal bacteria;

FIG. 25 is the effect of the probiotic compound microecological preparation on the relative abundance of species at the phylum level of vaginal fungi;

FIG. 26 is the effect of the probiotic compound microecological preparation on the relative abundance of species at the genus level of vaginal fungi.

### DETAILED DESCRIPTION

The following provides a further detailed description of the present disclosure with reference to the drawings.

### I, Experimental Methods

### 1, Medium and Solution Preparation

MRS liquid medium: The purchased MRS liquid medium (the formulation of the MRS liquid medium: 10 g of peptone, 10 g of beef extract, 5 g of yeast extract, 2 g of diammonium citrate, 5 g of sodium acetate, 20 g of glucose, 1 mL of Tween 80, 0.5 g of magnesium sulfate, 0.25 g of manganese sulfate, and 15 g of agar powder) is dissolved in 1 L of distilled water and sterilized at 121° C. for 15 min.

MRS solid medium: 15.0 g of agar is added to MRS liquid medium and sterilized at 121° C. for 15 min.

Yeast Mold (YM) solid medium: The purchased YM solid medium (the formulation of YM solid medium: 20 g of glucose, 5 g of peptone, 3 g of yeast extract powder, 1 g of potassium dihydrogen phosphate, 0.5 g of magnesium sulfate, 15 g of agar) is dissolved in 1 L of distilled water and sterilized at 121° C. for 15 min.

Yeast Extract Peptone Dextrose (YPD) liquid medium: The purchased YPD liquid medium (the formulation of YPD liquid medium: 10 g of yeast extract powder, 20 g of peptone, 20 g of glucose) is dissolved in 1 L of distilled water and sterilized at 121° C. for 15 min.

Selective Gardnerella vaginalis culture medium: The purchased selective Gardnerella vaginalis culture medium is dissolved in 1 L of distilled water and sterilized at 121° C. for 15 min. When in use, the selective Gardnerella vaginalis culture medium has been cooled to approximately 50° C., 5 wt %-10 wt % sterile defibrillated horse blood, 15-20 μg/mL of nalidixic acid solution, 10-20 μg/mL of gentamicin sulfate, and 2-5 μg/mL of amphotericin B solution are added.

Blood agar base medium: The purchased blood agar base medium (the formulation of blood agar base medium formula: 10 g of peptone, 3 g of beef extract, 5 g of sodium chloride, 15 g of agar) is dissolved in 1 L of distilled water and sterilized at 121° C. for 15 min. When in use, the above base medium has been cooled to approximately 50° C., 5 wt %-10 wt % sterile defibrinated sheep blood or rabbit blood is added to the above base medium to provide special nutrients for bacterial growth and help to observe the hemolysis of some bacteria.

### 2, Activation and Preservation of Bacterial Strains

2 mL of lactic acid bacteria strains preserved in a −80° C. freezer are selected and inoculated into 50 mL of fermentation medium, and cultured at 37° C. for 12 h to obtain the seed liquid. The seed liquid is then plated and cultured, and the colony morphology is observed, and the plates are inverted and stored in a freezer at −4° C. for future use.

### 3, Preparation of Lactic Acid Bacteria and Pathogenic Bacteria Solution

Culture of lactic acid bacteria: The above-mentioned preserved lactic acid bacteria seed liquid is inoculated into the fermentation medium (the MRS solid medium is dissolved in 1 L of distilled water, and sterilized at 121° C. for 15 min), cultured at 37° C. for 18 h, and activated for three generations to obtain lactic acid bacterial culture, and its optical density at 600 nm (OD₆₀₀) is adjusted to 1, and the bacterial liquid is prepared for subsequent use.

Cell-free supernatant (CFS) of lactic acid bacteria: The obtained lactic acid bacterial culture is centrifuged at 8000 r/min for 10 min, and the fermentation supernatant is filtered by a 0.22 μm polyethersulfone (PES) membrane to obtain CFS.

Culture of Gardnerella vaginalis: Gardnerella vaginalis is inoculated onto blood agar solid medium, after two generations of single colony activation, it is anaerobically cultured in selective Gardnerella vaginalis liquid medium at 37° C. for 24-48 h, and the concentration of bacteria is adjusted to 1×10⁸ CFU/mL.

Culture of Candida albicans: Candida albicans is inoculated onto YM solid medium, after two generations of single colony activation, it is cultured in YPD liquid medium at 30° C. for 24 h, and the cell concentration is adjusted to 4×10⁶ CFU/mL.

4, Determination of the Capability to Inhibit Gardnerella vaginalis and Candida albicans

In a 96-well plate, 150 μL of YPD liquid medium containing a 2% (v/v) inoculum of Candida albicans and 50 μL of the lactic acid bacteria CFS are added and mixed, the plate is incubated in a biochemical incubator at 30° C. for 24 h, and the OD₆₀₀ value is measured and recorded as the OD_{treatment group}; 200 μL of YPD medium containing a 2% (v/v) inoculum of Candida albicans is incubated at 30° C. for 24 h, and its OD₆₀₀ value is recorded as the OD_{control group}.

Separately, 150 μL of medium containing a 2% (v/v) inoculum of Gardnerella vaginalis and 50 μL of the lactic acid bacteria fermentation supernatant are added and mixed in an anaerobic incubator at 37° C. for 24-48 h, the OD₆₀₀ value is measured and recorded as the OD_{treatment group}; 200 μL of medium containing a 2% (v/v) inoculum of Gardnerella vaginalis is incubated at 37° C. for 24-48 h, and its OD₆₀₀ value is recorded as the OD_{control group}.

The calculation formula is as follows:

$$Inhibition rate ( \% ) = \frac{OD control group - OD tretament group}{OD control group} \times 100 \% . \qquad ( 1 )$$

### 5, Growth Curve and Acid Production Curve

After the seed liquid is activated for three generations, the seed liquid is inoculated and cultured at an inoculum size of 2% (v/v), and the pH value, along with the absorbance value at 600 nm of the fermentation broth, is measured every 2 h. The culture medium is fully shaken before each measurement, and three replicates are measured.

### 6, Determination of Acid and Bile Salt Tolerance

Acid tolerance is evaluated using MRS broth at pH 3.0 and 3.5, and bile tolerance is tested using MRS broth supplemented with 0.3%, 0.6%, and 0.9% (w/v) porcine bile salts. Five strains of lactic acid bacteria are inoculated into the above medium at an inoculum size of 2% (v/v), and cultured at 37° C. for 18 h, the OD₆₀₀ is measured and recorded as A1; the five strains of lactic acid bacteria are inoculated into MRS broth without additives and cultured at 37° C. for 18 h, the OD₆₀₀ is measured and recorded as A0. The survival rate calculation formula is as follows:

$$Survival rate ( \% ) = \frac{A 0 - A 1}{A 0} \times 100 \% . \qquad ( 2 )$$

### 7, Determination of Auto-Aggregation Capability and Surface Hydrophobicity

The bacterial liquid is inoculated into MRS broth medium at an inoculum size of 2% (v/v) and cultured for 18 h. The bacteria are collected by centrifugation at 5000 r/min for 10 min at 4° C., washed twice with PBS, and then resuspended in PBS, the OD₆₀₀ is measured, and the OD₆₀₀ of the bacterial suspension is adjusted to 0.25±0.05, and the initial OD is recorded as A0. A total of 4 mL of bacterial suspension is taken and transferred into an Eppendorf (EP) tube, after standing at 37° C. for 20 h, 3 mL of supernatant is added to another centrifuge tube to determine its OD₆₀₀, which is recorded as A1. The auto-aggregation rate is calculated according to the following formula. Three independent experiments are repeated, and the average value is taken.

$$Auto - aggregation rate ( \% ) = \frac{A 0 - A 1}{A 0} \times 100 \% ; \qquad ( 3 )$$

The bacterial suspension is prepared according to the above method, 1 mL of xylene is added to 3 mL of bacterial suspension, and the mixture is thoroughly mixed by vortex mixer, the water phase is taken after standing for 1 h at 37° C., and the OD₆₀₀ of the water phase is recorded as A. The hydrophobicity of the strain is calculated by the following formula. Three independent experiments are repeated, and the average value is taken.

$$Hydrophobicity ( \% ) = \frac{A 0 - A 1}{A 0} \times 100 \% . \qquad ( 4 )$$

### 8, Determination of Antibiotic Susceptibility

10 mL of MRS solid medium is poured into the Petri dish, and after solidification, soft agar containing 1% (v/v) inoculum of lactic acid bacteria is poured. After solidification, the antibiotic susceptibility disks are placed flat on the surface, and the group number is recorded. The antibiotic susceptibility disks used are: Vancomycin, tetracycline, streptomycin, penicillin, ampicillin, erythromycin, kanamycin, ceftazidime, gentamicin, and doxycycline. The Petri dishes containing antibiotic susceptibility disks are cultured at 37° C. for 48 h, and three replicates are set up to measure and record the size of the inhibition zone produced by each antibiotic susceptibility disk.

### 9, Transmission Electron Microscopy (TEM)

The fermented lactic acid bacterial culture is centrifuged at 5000 r/min for 10 min, and the bacterial pellet is collected and washed three times with PBS (pH 7.4) to remove the residual culture medium. Subsequently, the bacteria are resuspended in PBS, and an appropriate amount of bacterial suspension is added to a 200-mesh copper grid (carbon support film) and allowed to stand for 15 min to allow the bacteria to sediment adequately. After adsorption, filter paper is used to absorb excess liquid to avoid an overly thick sample. Then, the copper grid is immersed in a 2%-5% uranyl acetate-ethanol solution for 30 s, and the staining is repeated three times to enhance contrast. Finally, the morphology and ultrastructure of the bacteria are observed under TEM.

### 10, Hemolysis Experiment

Lactic acid bacteria are inoculated onto blood-containing agar plates suitable for the growth of the strains, and the hemolytic zone is observed.

11, 16S rDNA Identification and Phylogenetic Analysis of Lactic Acid Bacteria

### (1) DNA Extraction

The fresh bacterial culture is centrifuged at 8000 rpm for 1 min to obtain the bacteria, and the total DNA of the strain is extracted using a commercial kit.

### (2) PCR Amplification and Product Detection

The 16S rRNA gene is amplified using universal primers, and the primer sequences are as follows:

Forward primer 27F:
5′-AGAGAGTTTGATCCTGGCTCAG-3′;
Reverse primer 1495R:
5′-CTACGGCTACCTTGTTACGA-3′;

the PCR cycle parameters are as follows: 94° C. pre-denaturation for 5 min; 34 cycles of denaturation at 94° C. for 1 min, annealing at 64° C. for 1 min, and extension at 72° C. for 2 min; and final extension at 4° C. for 10 min. The amplified PCR products are sent to Sangon Biotech (Shanghai) Co., Ltd. for gene sequencing.

### (3) Phylogenetic Tree

The obtained strain sequence is aligned with the 16S rDNA gene fragment on the National Center for Biotechnology Information (NCBI) website, and the 16S rDNA gene sequence of the strain with high similarity is extracted, and the phylogenetic tree is constructed using the software MEGAX 64.

### 12, Optimization of the Compounding Ratio of the Probiotic Microecological Preparations

The three selected strains of lactic acid bacteria are activated and their OD₆₀₀ are adjusted to 1 (10⁹ CFU/mL), they are then compounded according to the ratios of 1:1:1, 1:1:2, 2:2:3, 3:2:1, 1:3:2, 1:1:0, 2:1:1, 3:1:1, 3:1:2, 3:2:2, 2:1:3, 1:0:1, 1:2:0, 2:0:1, 1:2:3, 1:2:1, 1:3:1, 2:3:2, 2:3:1, 0:1:1, 0:1:2, 1:0:2, 0:2:1, 2:1:0, each compounded preparation is inoculated at 2% (v/v) into 10 mL of MRS broth. After incubation for 18 h, the culture is centrifuged at 8000 r/min for 10 min, and the fermentation supernatant is collected and filtered through a 0.22 μm PES membrane to obtain the CFS.

In a 96-well plate, 150 μL of YPD liquid medium containing a 2% (v/v) inoculum of Candida albicans and 50 μL of the lactic acid bacteria CFS are added and mixed, the plate is incubated in a biochemical incubator at 30° C. for 24 h, and the OD₆₀₀ value is measured and recorded as the OD_{treatment group}; 200 μL of YPD medium containing a 2% (v/v) inoculum of Candida albicans is incubated at 30° C. for 24 h, and its OD₆₀₀ value is recorded as the OD_{control group}.

Separately, 150 μL of medium containing a 2% (v/v) inoculum of Gardnerella vaginalis and 50 μL of the lactic acid bacteria fermentation supernatant are added and mixed in an anaerobic incubator at 37° C. for 24-48 h, the OD₆₀₀ value is measured and recorded as the OD_{treatment group}; 200 μL of medium containing a 2% (v/v) inoculum of Gardnerella vaginalis is incubated at 37° C. for 24-48 h, and its OD₆₀₀ value is recorded as the OD_{control group}.

$$Inhibition rate ( \% ) = \frac{OD control group - OD treatment group}{OD control group} \times 100 \% , \qquad ( 1 )$$

By comparing the inhibition rates of the two, the compounding ratio with the greatest potential for improving vaginitis is selected.

### 13, Detection of Freeze-Drying Survival Rate of Probiotics Microecological Preparation

The three selected lactic acid bacteria are mixed according to the best compounding ratio, the compounded preparation is inoculated at 2% (v/v) into MRS broth and cultured for 18 h, after incubation, the culture is centrifuged at 8000 r/min for 10 min to obtain bacterial pellets, 2 mL of lyoprotectant is added to each 50 mL of culture obtained by centrifugation, and freeze-dried to obtain a high concentration of lactic acid bacteria preparation.

The lyoprotectant is the optimal formula after the previous optimization in the laboratory: 118.2 g/L of trehalose, 17.1 g/L of L-cysteine, 10.3 g/L of sorbitol, 1.7 g/L of sodium acetate, and 120 g/L of skim milk powder. The bacterial suspension and powder before and after freeze-drying are plated and enumerated by the ten-fold gradient dilution method, and the freeze-drying survival rate is calculated by dividing the viable count after freeze-drying by that before freeze-drying.

### 14, Detection of the Aerobic Plate Count of Probiotics Microecological Preparation During Storage

The freeze-dried bacterial powder is stored at 4° C., 37° C., and room temperature (10° C.-20° C.). On the 0, 7, 14, 21, and 28 days of storage, the microecological preparations under different storage conditions are diluted and enumerated.

### 15, Data Processing and Statistical Analysis.

Each experiment is repeated three times. GraphPad Prism 9.4.0 and SPSS 19.0 are used to analyze the experimental data and generate figures, the experimental results are in the form of mean±standard deviation (mean±SEM). The significance of multiple sets of data is analyzed by one-way analysis of variance (ANOVA).

### II, Screening of Strains

### 1, Screening of Lactic Acid Bacteria

When the vaginal microecological balance is broken, harmful bacteria such as Gardnerella vaginalis and Candida albicans will multiply. Lactic acid bacteria can produce lactic acid, H₂O₂, bacteriocins, and other substances, effectively inhibit the growth and reproduction of harmful bacteria, and help restore the balance of vaginal microecology. In the process of applying lactic acid bacteria to the treatment of vaginitis, antibacterial activity is the most critical index, which can be used as the primary standard for screening lactic acid bacteria with the potential to improve vaginitis. In this study, a total of 32 lactic acid bacteria strains are randomly selected from the strain collection bank of the Animal Product Processing Laboratory at Ningbo University for antibacterial assays, by evaluating the inhibitory capability of their CFS against Gardnerella vaginalis and Candida albicans, five lactic acid bacteria strains with the potential to alleviate vaginitis are selected.

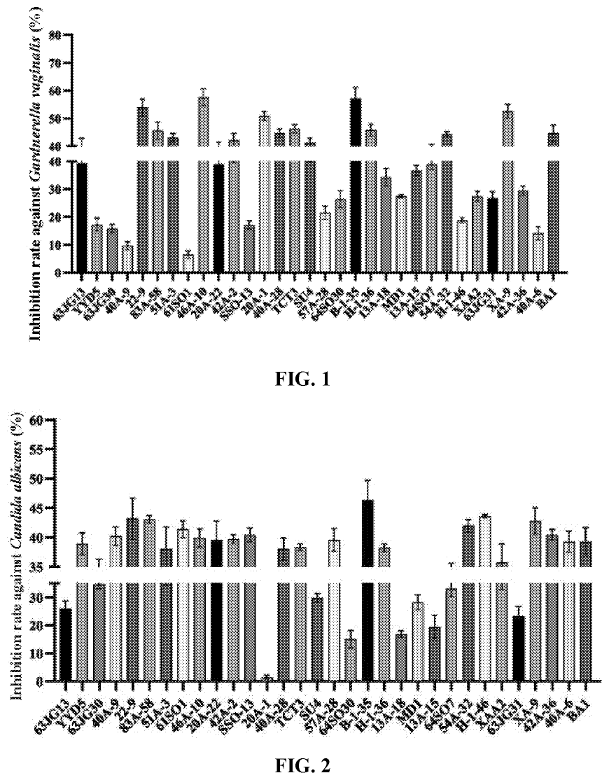

From FIG. 1, the growth inhibition rates of Gardnerella vaginalis by lactic acid bacteria B-1-35, 22-9, XA-9, and 20A-1 are 57.22%, 53.96%, 52.58%, and 50.87%, respectively. From FIG. 2, the growth of Candida albicans is best inhibited by B-1-35, 83A-58, H-1-46, XA-9, 54A-32, and 22-9, which are 46.34%, 43.09%, 43.61%, 42.78%, 41.97%, and 43.18%, respectively. Based on the inhibitory capability of lactic acid bacteria to Gardnerella vaginalis and Candida albicans, lactic acid bacteria 22-9, 83A-58, B-1-35, 54A-32, and XA-9 are selected as candidate strains of compound microecological preparations.

### 2, Growth Curve and Acid Production Curve

By plotting the growth curve, the growth status of lactic acid bacteria in different culture stages can be determined, which is helpful to determine the optimal culture time of lactic acid bacteria and provide an experimental basis for the production of microecological preparations. The acid production curve can reflect the acid production capability and dynamic changes of lactic acid bacteria during the growth process, and can be used to determine the acid production capability of different lactic acid bacteria strains.

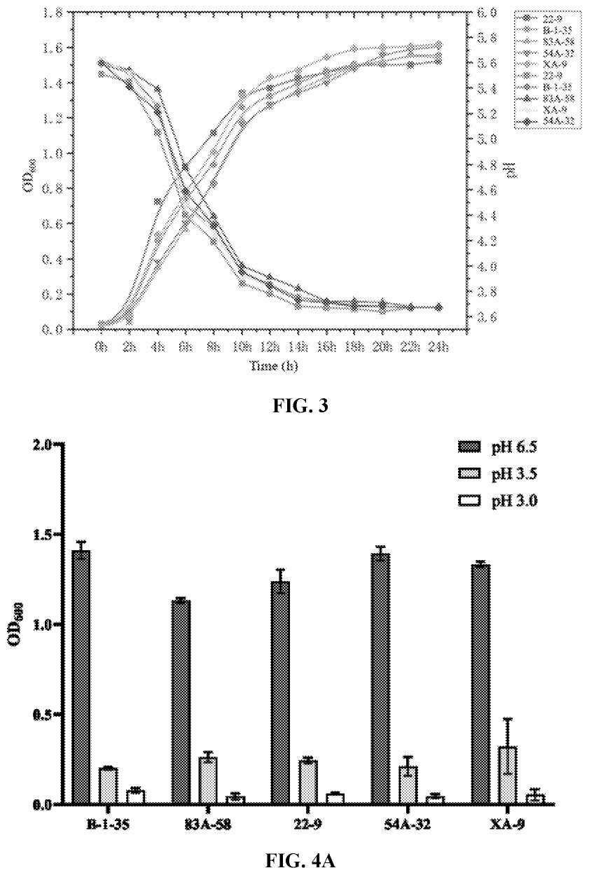

As shown in FIG. 3, when the culture temperature is 37° C., the 5 strains of lactic acid bacteria are adjusted to the same concentration, inoculated into MRS liquid medium with 2% (v/v), and entered the logarithmic growth phase after 2 h and reached the stationary phase after 14 h; the initial pH of the lactic acid bacteria fermentation broth is approximately 5.60, which rapidly decreased to around 3.80 within 2-10 h of cultivation, followed by a slow decline that stabilized after 14 h of inoculation. The growth curves and pH trends of the five lactic acid bacteria strains are consistent, with no significant differences observed. The experimental results indicate that all the above strains possess strong acid production capacity and may rapidly enter the logarithmic phase, leading to a swift decrease in pH, this characteristic is beneficial for restoring the acidic vaginal environment and inhibiting the growth of pathogenic bacteria.

### 3, Determination of Acid and Bile Salt Tolerance

The human gastrointestinal tract presents a complex environment, with gastric acid and bile salts being crucial components. Detecting the acid and bile salt tolerance of lactic acid bacteria simulates their survival conditions within the gastrointestinal tract, allowing for the evaluation of whether lactic acid bacteria can maintain their activity and exert beneficial effects after passing through the acidic stomach environment and the bile salt conditions of the small intestine. In the development of microecological preparations, selecting lactic acid bacteria strains with strong acid and bile salt tolerance ensures that a sufficient number of viable bacteria reach the intestine after oral administration, thereby enhancing the efficacy and stability of the lactic acid bacteria preparation.

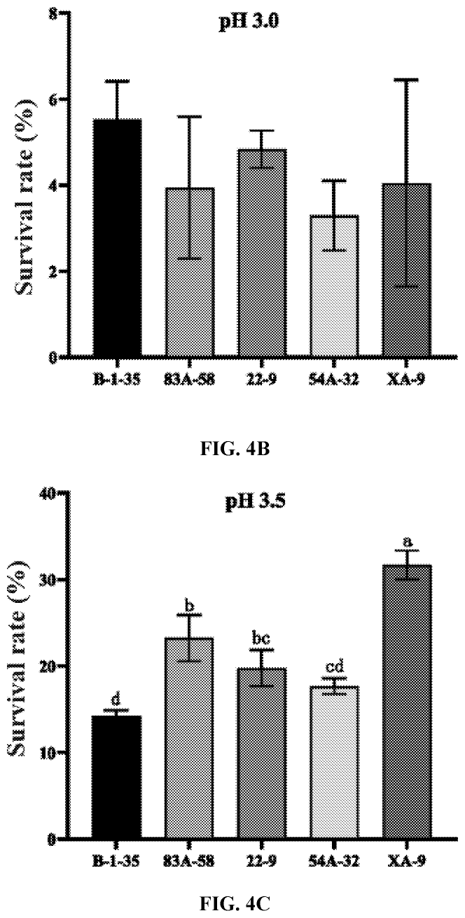

As shown in FIG. 4A, when the lactic acid bacteria grow in the environment of pH 6.5 (control), the lactic acid bacteria grow rapidly, and the OD₆₀₀ is above 1.1. With the decrease in pH value, the growth capability of each lactic acid bacterium changed. As shown in FIG. 4C, the survival rates of B-1-35, 83A-58, 22-9, 54A-32 and XA-9 are 14.23%, 23.23%, 19.71%, 15.16% and 24.12% respectively at pH 3.5. At this time, the survival rate of XA-9 is the highest, which is significantly (P<0.05) higher than that of the other strains. As shown in FIG. 4B, the survival rate of lactic acid bacteria decreased significantly at pH 3, which are 5.55%, 3.93%, 4.83%, 3.29% and 4.03%, respectively. There is no significant difference between the five strains. In conclusion, these results indicate that all tested lactic acid bacteria strains possess a certain degree of tolerance to acidic conditions.

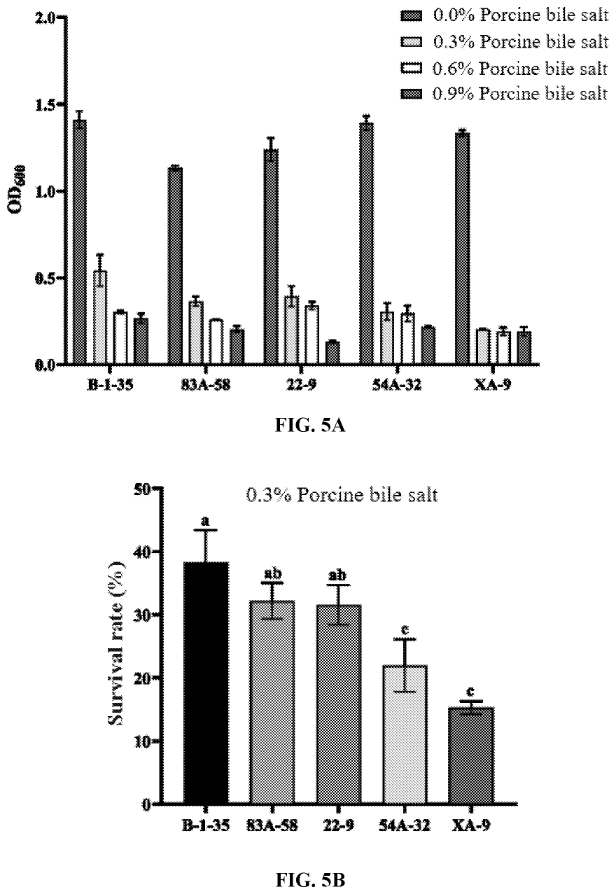

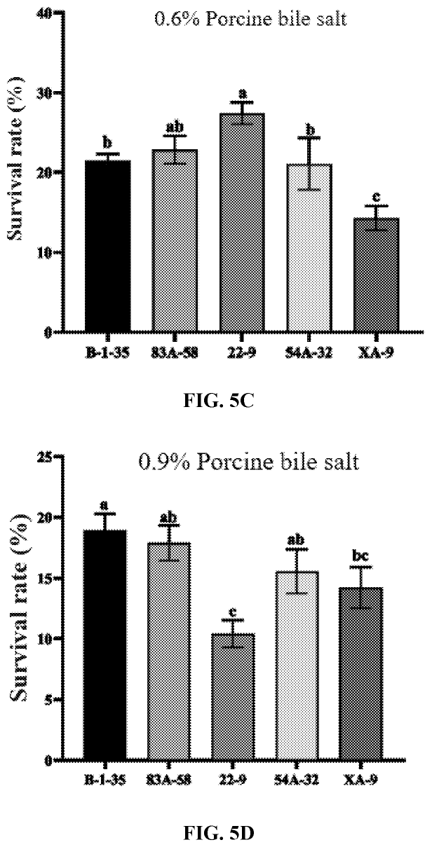

As shown in FIG. 5A, the lactic acid bacteria grow normally when the bile salt concentration is 0%, and the growth value reaches the maximum. With the increase in bile salt concentration, the growth capability of each lactic acid bacterium changed. As shown in FIG. 5B, the survival rates of B-1-35, 83A-58, 22-9, 54A-32, and XA-9 are 38.31%, 32.20%, 31.57%, 21.98% and 15.28%, respectively, when the bile salt concentration is 0.3%. At this time, the survival rate of B-1-35 is the highest, which is significantly (P<0.05) higher than that of 54A-32 and XA-9. As shown in FIG. 5C, when the concentration of bile salt is 0.6%, the survival rates of lactic acid bacteria are 21.46%, 22.85%, 27.43%, 21.08% and 14.28%, respectively; where 54A-32 and XA-9 show almost no change, and 22-9 decreases slightly. As shown in FIG. 5D, when the bile salt concentration is 0.9%, the survival rates of the lactic acid bacteria are 18.89%, 17.88%, 10.42%, 15.55%, and 14.21%, respectively; the survival rates of lactic acid bacteria do not decrease significantly. In conclusion, these results indicate that the above lactic acid bacteria have a certain tolerance to the bile salt environment.

### 4, Determination of Auto-Aggregation Capability and Surface Hydrophobicity

The auto-aggregation capability helps lactic acid bacteria to build communities, better resist antibacterial substances, and maintain high density in the local environment. At the same time, the auto-aggregation capability of lactic acid bacteria is closely related to their adhesion characteristics; lactic acid bacteria with strong auto-aggregation capability are more likely to colonize in the vagina. Hydrophobicity affects the interaction with other substances in the surrounding environment. Lactic acid bacteria with suitable hydrophobicity can better specifically or non-specifically bind to epithelial cells and other microbial cells, which is conducive to the colonization of lactic acid bacteria in the human body. The auto-aggregation of lactic acid bacteria is about 16-35% for low auto-aggregation capability, 36-50% for medium auto-aggregation capability, and more than 51% for high auto-aggregation capability. The cell adhesion capability of lactic acid bacteria plays an extremely important role in both oral and vaginal administration. Taking auto-aggregation capability and surface hydrophobicity as the second screening criteria, combined with the characteristics of acid and bile salt tolerance of lactic acid bacteria, three strains are selected from five strains for the preparation of probiotics microecological preparation.

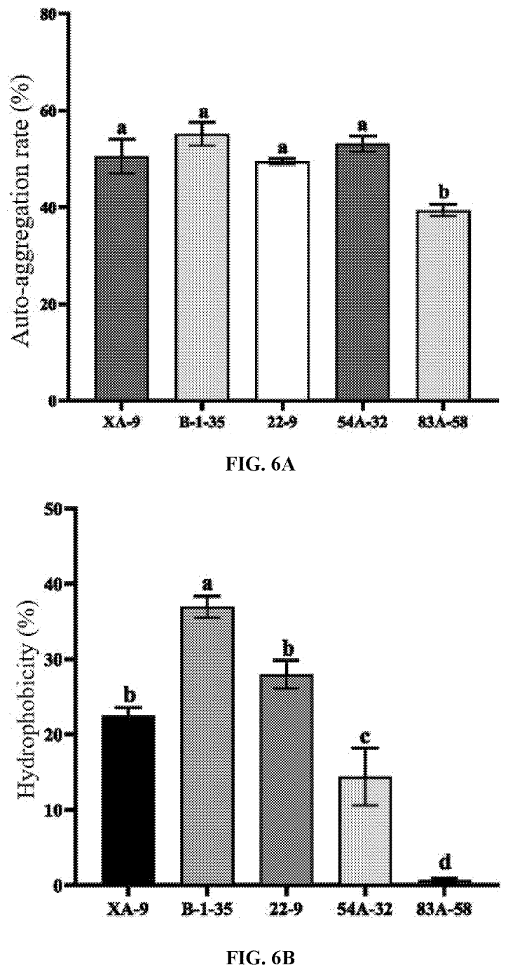

According to FIG. 6A, the auto-aggregation rates of XA-9, B-1-35, 22-9, and 54A-32 are 52.52%, 55.14%, 51.42% and 53.09%, respectively, all of which exhibit high auto-aggregation capability, and 83A-58 has medium auto-aggregation capability. According to FIG. 6B, it can be seen that XA-9, B-1-35 and 22-9 are moderately hydrophobic, and 54A-32 and 83A-58 are non-hydrophilic. Auto-aggregation and surface hydrophobicity are important indicators for evaluating the cell adhesion capability of lactic acid bacteria. Therefore, based on these two factors, combined with their tolerance to acid and bile salts, the component strains for the microecological preparation are screened. Ultimately, XA-9, B-1-35, and 22-9 are selected as the constituent strains for the microecological preparation.

### 5, Determination of Antibiotic Susceptibility

The purpose of testing the antibiotic susceptibility of lactic acid bacteria is to prevent the transfer of resistance genes and to avoid interference when subsequently used in combination with drug therapy. Drug-resistant lactic acid bacteria may carry resistance genes, which could potentially be transferred to some pathogenic bacteria harmful to the human body. Testing antibiotic susceptibility helps to promptly detect and control the spread of resistant strains, preventing the dispersal of resistance genes within the microbial community.

Meanwhile, in the treatment of vaginitis, a combined approach using antibiotics and lactic acid bacteria is often adopted. Testing the antibiotic susceptibility of lactic acid bacteria facilitates the selection of drugs that are effective against pathogens while having minimal impact on lactic acid bacteria during combination therapy. This improves the safety and efficacy of the treatment, avoiding interference from antibiotic therapy with the therapeutic effects of lactic acid bacteria.

TABLE 1
Antibiotic susceptibility test results of lactic acid bacteria

| | Lactic acid bacteria | | |
|---|---|---|---|
| Antibiotic susceptibility disk | 22-9 | B-1-35 | XA-9 |
| Vancomycin | —(R) | —(R) | —(R) |
| Tetracycline | 22.83 ± 0.76(S) | 19.5 ± 0.71(S) | 23.5 ± 0.5(S) |
| Streptomycin | 8.25 ± 0.35(R) | —(R) | 9.83 ± 0.71(R) |
| Penicillin | 26.33 ± 1.15(R) | 26 ± 1(R) | 27.33 ± 0.58(R) |
| Ampicillin | 30.33 ± 1.53(S) | 30.33 ± 0.58(S) | 33.83 ± 1.89(S) |
| Erythromycin | 25.67 ± 1.15(S) | 24.67 ± 1.53(S) | 26 ± 1(S) |
| Kanamycin | 8.33 ± 0.58(R) | —(R) | 10 ± 1(R) |
| Ceftazidime | 21.33 ± 2.08(S) | 30 ± 1(S) | 21 ± 1(S) |
| Gentamicin | 14.83 ± 0.29(R) | 12.83 ± 0.76(I) | 16.17 ± 0.29(S) |
| Doxycycline | 27.33 ± 0.58(S) | 25.33 ± 1.15(S) | 27 ± 1(S) |

Note:
Interpretation criteria (diameter of inhibition zone): S denotes susceptibility, I denotes intermediate, R denotes resistance.

According to Table 1, the susceptibility of lactic acid bacteria strains 22-9, B-1-35, and XA-9 to antibiotics is almost identical, and all three strains are resistant to vancomycin, streptomycin, penicillin, and kanamycin; sensitive to tetracycline, ampicillin, erythromycin, ceftazidime, and doxycycline; differences are only observed in their response to gentamicin: strain 22-9 is resistant, strain B-1-35 shows intermediate susceptibility, and strain XA-9 is susceptible.

### 7, Hemolysis Experiment

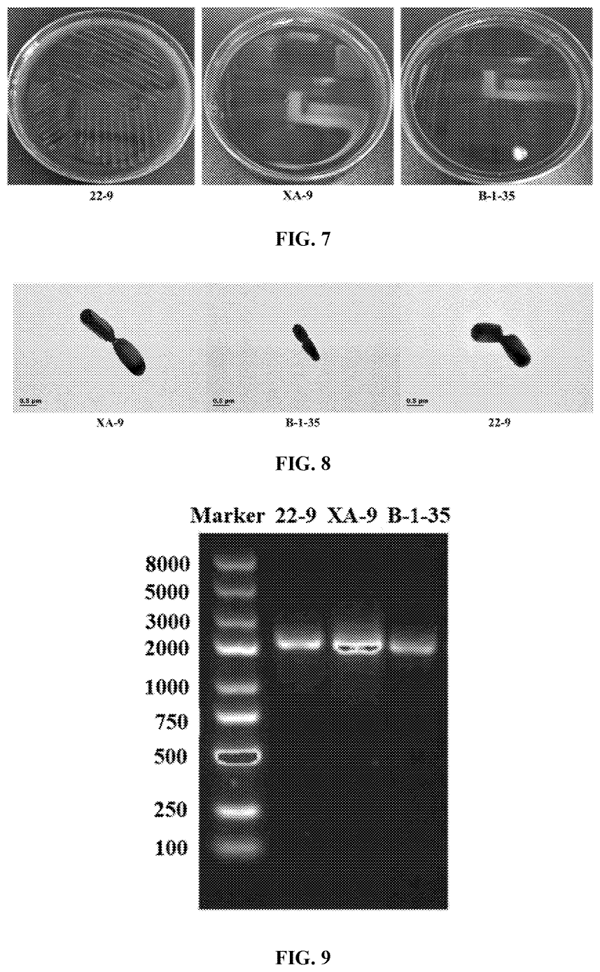

Hemolytic capability is an important index to measure the potential pathogenicity of bacteria. Some lactic acid bacteria with hemolytic properties may cause damage to host cells, trigger inflammatory reactions, and even lead to diseases. Through the hemolysis experiment, it can be preliminarily judged whether lactic acid bacteria have potential pathogenicity. The experimental results, as shown in FIG. 7, demonstrate that none of the tested strains exhibited hemolytic phenomena on blood agar plates.

### 8, Transmission Electron Microscopy (TEM)

The cell morphology and structure of different lactic acid bacteria are different, the fine structure and overall shape of the cell wall and cell membrane can be observed by a transmission electron microscope, which can assist in classification and identification. The morphology reflects the physiological state, under different growth stages and culture conditions, the morphology of lactic acid bacteria is different, so that the functional changes of metabolism, growth, and stress response can be speculated.

As shown in FIG. 8, all three strains are rod-shaped bacteria. XA-9 appears to be in the division stage, exhibiting a dumbbell shape, with what may be a new cell wall or membrane structure in the middle connecting the daughter cells; B-1-35 is short and compact, with clear boundaries and no signs of division, B-1-35 may be in the logarithmic growth phase, characterized by stable cell status and intact structure; 22-9 shows two cells connected, possibly having just divided but not yet completely separated.

### 9, Identification Results of Lactic Acid Bacteria Strains

(1) Amplification of 16S rDNA of Lactic Acid Bacteria Isolates

The gel electrophoresis results of the PCR amplification products are shown in FIG. 9. The products have no degradation, no tailing phenomenon, good integrity, and meet the test requirements. The left side of FIG. 9 is the DNA marker, with a maximum of 8000 bp, and the remaining three columns from left to right are strain 22-9, XA-9, and B-1-35, respectively.

### (2) Phylogenetic Tree and Homology Analysis

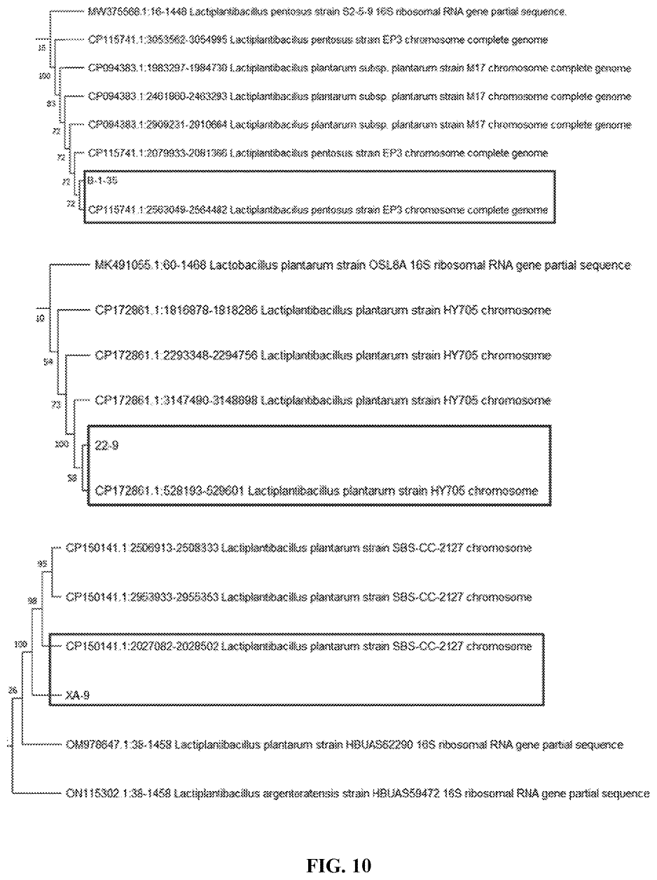

The phylogenetic tree of strains 22-9, XA-9, and B-1-35 is shown in FIG. 10, as shown in FIG. 10 that strains 22-9 and XA-9 are Lactiplantibacillus plantarum, and B-1-35 is Lactiplantibacillus pentosus.

In summary, 32 strains of lactic acid bacteria are randomly selected from the Ningbo strain library for screening, and 5 strains of lactic acid bacteria that significantly inhibited the growth of Gardnerella vaginalis and Candida albicans are obtained. On this basis, using the auto-aggregation capability and surface hydrophobicity index to evaluate the adhesion capability of lactic acid bacteria, three lactic acid bacteria strains with excellent antibacterial properties are identified, which are 22-9, XA-9, and B-1-35, respectively.

The growth curves and acid production curves indicate that all three strains are capable of rapid growth and stable acid production, they enter the exponential growth phase after 2 h and maintain a prolonged stationary phase, demonstrating good growth stability. The three strains exhibit excellent tolerance to acidic and bile salt environments, where strain XA-9 shows the best tolerance, achieving a growth rate of 24.12% at pH 3.5. Meanwhile, the auto-aggregation rates of XA-9, B-1-35, and 22-9 are 52.52%, 55.14%, and 51.42%, respectively, indicating high auto-aggregation ability, and all three strains are moderately hydrophobic. In vitro safety evaluation shows that the three strains meet the safety criteria and could be used as composite strains for probiotic microecological preparations. Through 16S rDNA sequencing identification, strains XA-9 and 22-9 are determined to be Lactiplantibacillus plantarum, while strain B-1-35 is identified as Lactiplantibacillus pentosus.

The Lactiplantibacillus plantarum strain 22-9, with the deposit number CGMCC No. 33867, is deposited at the China General Microbiological Culture Collection Center (CGMCC) on Mar. 19, 2025, and a preservation address is No. 3, No. 1 West Beichen Road, Chaoyang District, Beijing.

The Lactiplantibacillus plantarum XA-9 strain, with the deposit number CGMCC No. 33868, is deposited at the CGMCC on Mar. 19, 2025, and the preservation address is No. 3, No. 1 West Beichen Road, Chaoyang District, Beijing.

The Lactiplantibacillus pentosus B-1-35 strain, with the deposit number CGMCC No. 33870, is deposited at the CGMCC on Mar. 19, 2025, and the preservation address is No. 3, No. 1 West Beichen Road, Chaoyang District, Beijing.

### III, Determination of Optimal Strain Ratio and Quality Testing of Probiotics Microecological Preparation

1, Effects of Different Fermentation Strains and Ratios on the Inhibitory Capability Against Gardnerella vaginalis and Candida albicans

Developing a microecological preparation composed with three lactic acid bacteria strains offers advantages over single-strain preparations: diverse functionality: containing a variety of bacteria, its metabolites and functions are different, can play a variety of roles; enhanced stability: the growth and metabolism of lactic acid bacteria are complementary, and the coexistence of multiple bacteria adapts to the complex environment and is easier to colonize; broader applicability: a variety of bacteria can target different vaginitis pathogens, and the scope of application is greater than that of single bacteria. Probiotic microecological preparations are safer than live bacterial preparations, after freeze-drying, lactic acid bacteria are in a dormant state to reduce the risk of death and mutation of lactic acid bacteria; they are also more durable; under suitable conditions in vivo, the bacteria slowly resuscitate, colonize, and reproduce, leading to prolonged effects, whereas viable bacteria are easily cleared and struggle to colonize long-term.

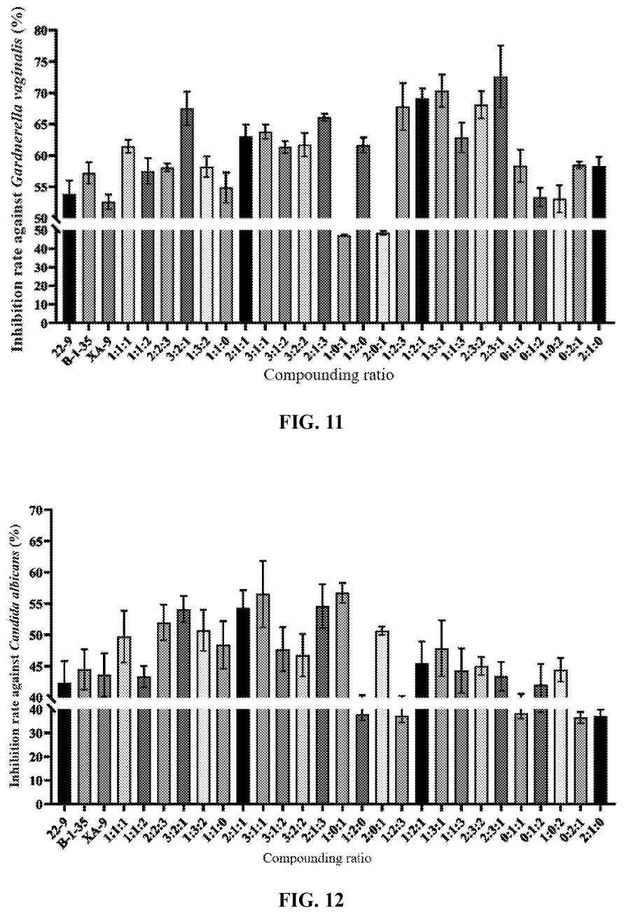

As shown in FIG. 11 that the inhibition rates of 22-9, XA-9, and B-1-35 against Gardnerella vaginalis at the same concentration are as follows: 53.83%, 52.58%, 57.23% respectively. When the proportion of 22-9, XA-9 and B-1-35 is 3:2:1, 2:1:3, 1:2:3, 1:2:1, 1:2:1, 1:3:1, 2:3:2, and 2:3:1, the inhibition rates are 67.52%, 66.11%, 67.82%, 69.13%, 70.35%, 68.10%, and 72.59%, respectively, the antibacterial activity is much higher than that of a single strain.

As shown in FIG. 12 that the inhibition rates of 22-9, XA-9, and B-1-35 against Candida albicans are 42.33%, 44.46%, and 43.58%, respectively. When the ratio of 22-9, XA-9 and B-1-35 is 1:0:1, 3:1:1, 2:1:3, 3:2:1, and 2:1:1, the inhibition rates are 56.73%, 56.51%, 54.59%, 54.08%, and 54.33%, respectively, the antibacterial activity is much higher than that of a single strain.

In summary, when the compounding ratios of 22-9, XA-9, and B-1-35 are 3:2:1 and 2:1:3, the inhibitory capacity of the compounded microecological preparation against Candida albicans and Gardnerella vaginalis is significantly higher than that of single strains. By comparison, the ratio of 2:1:3 is selected as the final ratio for the microecological preparation.

### 2, Detection of Freeze-Drying Survival Rate of Probiotics Microecological Preparation

The detection of freeze-drying survival rate of microecological preparations is helpful to intuitively understand the impact of freeze-drying on lactic acid bacteria and to judge the effect of freeze-drying. At the same time, the survival rate is related to the quality and efficacy of microecological preparations; only with a sufficient number of surviving viable bacteria can the microecological balance be effectively regulated, harmful bacteria be inhibited, and the desired effects be achieved.

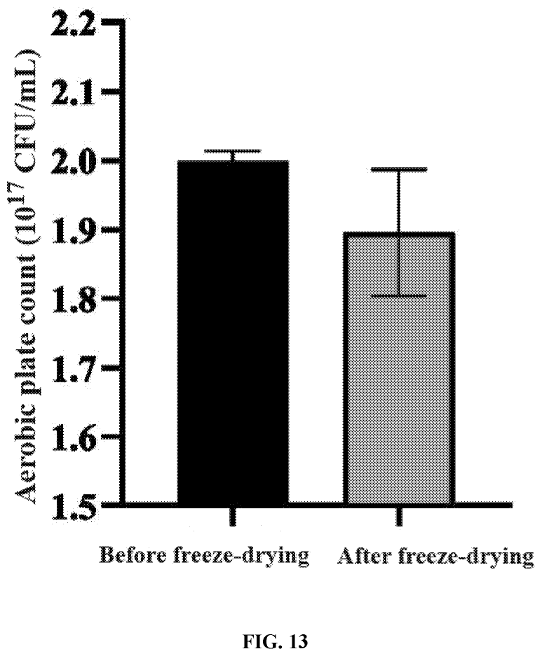

As shown in FIG. 13 that the aerobic plate count before and after freeze-drying of undiluted microecological preparations is in the order of 10¹⁷ CFU/mL, and the freeze-drying survival rate reached 94.80%.

### 3, Detection of Aerobic Plate Count in Probiotic Microecological Preparations During Storage

The detection of the aerobic plate count during the storage period of microecological preparations is designed to control the quality, the aerobic plate count directly reflects the number of microorganisms. An abnormal increase in the count may indicate potential contamination by foreign bacteria or uncontrolled microbial growth, thereby disrupting the microecological balance and affecting product quality. Additionally, this detection helps evaluate stability. Regular detection based on changes in the aerobic plate count allows for the assessment of product stability and the determination of optimal storage conditions.

As shown in FIG. 14 that the aerobic plate count of the undiluted microecological preparation remained largely unchanged when stored at 4° C. When stored at room temperature (10-20° C.), the aerobic plate count shows a slight decreasing trend, while at 37° C., the lactic acid bacteria count decreases significantly. Despite this, the aerobic plate count of lactic acid bacteria in the microecological preparation remained relatively high. However, it is uncertain whether the activity of lactic acid bacteria in a dormant state will be affected, storage at 4° C. is recommended.

### IV, Product Application Embodiment One

### 1, Specific Embodiments

### Embodiment 1

A method for preparing a probiotic compound microecological preparation with efficacy in improving vaginitis includes the following steps:
(1) Lactic acid bacteria fermentation: The lactic acid bacterial cultures of Lactiplantibacillus plantarum 22-9, Lactiplantibacillus plantarum XA-9, and Lactiplantibacillus pentosus B-1-35 are mixed at a volume-to-volume ratio (v/v) of 2:1:3 to obtain a mixed starter, the mixed starter is inoculated into the fermentation medium at a volume ratio of 2% and cultured at 37° C. for 18 h to obtain a mixed culture of lactic acid bacteria.
(2) Freeze-drying: The mixed culture of lactic acid bacteria is centrifuged at 8000 rpm, at 4° C. for 15 min, and the lactic acid bacteria pellet is taken, the bacterial concentration is adjusted to 1× 10⁹ CFU/mL by sterile PBS, and the lyoprotectant solution of the same volume as sterile PBS is added. The mixture is put into the freezer at −80° C. overnight for pre-freezing, the pre-frozen sample is freeze-dried at −49° C. and 9 Pa for 48 h to obtain a probiotic compound microecological preparation. The formula of lyoprotectant is 118.2 g/L of trehalose, 17.1 g/L of L-cysteine, 10.3 g/L of sorbitol, 1.7 g/L of sodium acetate, and 120 g/L of skim milk powder.

### (3) Mode of Administration: Vaginal Administration

The final prepared compound microecological preparation is named the high-dose vaginal administration group (HD).

### Embodiment 2

This embodiment is the same as Embodiment 1 above, with the difference being that in step (3), the administration method is oral and vaginal administration. The final prepared compound microecological preparation is named the high-dose combined administration (oral+vaginal administration) group (HH).

### Embodiment 3

This embodiment is the same as Embodiment 1 above, with the difference being: in step (2), the lactic acid bacteria pellet is adjusted to 1× 10⁸ CFU/mL using sterile PBS. The final prepared compound microecological preparation is named the middle-dose vaginal administration group (MD).

### Embodiment 4

This embodiment is the same as Embodiment 3 above, with the difference being: in step (3), the administration method is oral and vaginal administration. The final prepared compound microecological preparation is named the medium-dose combined administration (oral+vaginal administration) group (MH).

### Embodiment 5

This embodiment is the same as Embodiment 1 above, with the difference being: in step (2), the lactic acid bacteria pellet is adjusted to 1×10⁷ CFU/mL using sterile PBS. The final prepared compound microecological preparation is named the low-dose vaginal administration group (LD).

### Embodiment 6

This embodiment is the same as Embodiment 5 above, with the difference being that in step (3), the administration method is oral and vaginal administration. The final prepared compound microecological preparation is named the low-dose combined administration (oral+vaginal administration) group (LH).

### 2, the Improvement Effect of Probiotics Compound Microecological Preparation on the Mouse Model of Vaginitis

1) Experimental materials: Specific pathogen-free (SPF) healthy, female Kunming mice (KM mice), weighing approximately 35.00±2 g each. Provided by SiPeiFu (Suzhou) Biotechnology Co., Ltd., Production License: SCXK (Su) 2022-0006, Batch Certificate Number: A202412190019.
2) Experimental environment and ethics: Carried out in the ABSL-2 laboratory of LenSci Biotechnology (Kunshan) Co., Ltd., laboratory animal license: SYXK (Su) 2023-0083; Suzhou Animal Pathogenic Microbiology Laboratory Registration No.: Kunming, Suzhou, Animal Laboratory for 320583012; Institutional Animal Care and Use Committee (IACUC) number: 20241216-0002-01.

Feeding conditions: Animals are raised in the experimental environment for 3 days after arrival. Animals are housed in independent ventilation cages (IVC) (5 per cage) in an ABSL-2 animal room. All cages, padding, and drinking water are sterilized before use. All experimenters wear protective clothing and latex gloves when operating in the animal room. Each cage animal information card indicate the number of animals in the cage, gender, strain, date of receipt, project number, and group. Feed and water are changed once a week. During the feeding period, the animals are free to eat feed and water. Temperature: 20-26° C.; humidity: 40-70%; light cycle: 12 h light, 12 h no light.

Feed Ingredients: Feed complies with the identification standards for experimental animals, from Keao Xieli (Tianjin) Feed Co., Ltd., Feed Production License No.: Jin Tiao Zheng (2020)01005, Implementation Standard: GB14924.3-2010; Maximum contaminant levels are maintained within acceptable limits and routinely inspected by the manufacturer; drinking water is produced by reverse osmosis filtration.
3) Experimental scheme: 88 mice are randomly divided into 8 groups; they are randomly assigned to 11 experimental groups, each group contains 8 mice. The experimental design used two different administration methods: vaginal single-channel administration (vaginal administration) and dual-channel combined administration (vaginal administration combined with intragastric administration). The specific grouping is set as follows: blank control group (CK), estradiol control group (CKC), vaginal administration model group (MDD), combined administration model group (MDH), commercially available lactic acid bacteria preparation group (CG), low-dose vaginal administration group (LD), low-dose combined administration group (LH), medium-dose vaginal administration group (MD), medium-dose combined administration group (MH), high-dose vaginal administration group (HD), and high-dose combined administration group (HH).

The whole experimental process can be divided into three stages: The induction of estrus (Day 1-6), the modeling period (Day 7-11), and the administration period (Day 12-19).

During the estrous induction period (Day 1-6), in addition to the CK group, each mouse is subcutaneously injected with estradiol benzoate (2 mg/mL), 0.05 mL, once every other day, until the end of the test.

During the modeling period (Day 7-11), 20 μL (10⁶ CFU/mL) of mixed bacterial suspension of Candida albicans and Gardnerella vaginalis is inoculated into the vagina of mice, and the vaginal orifice of mice is gently pinched with small tweezers for 30 s to prevent the leakage of bacterial suspension. The CK group and the CKC group are given the same amount of normal saline for 5 days, the appearance of the vaginal orifice is observed, and the vaginal lavage fluid is taken to detect whether the modeling is successful according to the Gram staining method.

During the administration period (Day 12-19), the dosing regimen (including route of administration and dose) of each group is shown in Table 2.

TABLE 2
Administration route and dose

| | | Vaginal | Administration | Intragastric | Administration |
|---|---|---|---|---|---|
| | Group | administration | dose | administration | dose |
| CK | Blank control | — | — | — | — |
| | group | | | | |
| CKC | Estradiol control | — | — | — | — |
| | group | | | | |
| MDD | Vaginal | ✓ | 20 μL PBS | — | 200 μL PBS |
| | administration | | | | |
| | model group | | | | |
| MDH | Combined | ✓ | 20 μL PBS | ✓ | 200 μL PBS |
| | administration | | | | |
| | model group | | | | |
| CG | Commercially | ✓ | 20 μL 10⁷ | — | — |
| | available lactic | | | | |
| | acid bacteria | | | | |
| | preparation group | | | | |
| LD | Low-dose | ✓ | 20 μL 10⁷ | — | — |
| | vaginal | | | | |
| | administration | | | | |
| LH | Low-dose | ✓ | 20 μL 10⁷ | ✓ | 20 μL 10⁷ |
| | combined | | | | |
| | administration | | | | |
| | group | | | | |
| MD | Medium-dose | ✓ | 20 μL 10⁸ | — | — |
| | vaginal | | | | |
| | administration | | | | |
| | group | | | | |
| MH | Medium-dose | ✓ | 20 μL 10⁸ | ✓ | 20 μL 10⁸ |
| | combined | | | | |
| | administration | | | | |
| | group | | | | |
| HD | High-dose vaginal | ✓ | 20 μL 10⁹ | — | — |
| | administration | | | | |
| | group | | | | |
| HH | High-dose | ✓ | 20 μL 10⁹ | ✓ | 20 μL 10⁹ |
| | combined | | | | |
| | administration | | | | |
| | group | | | | |

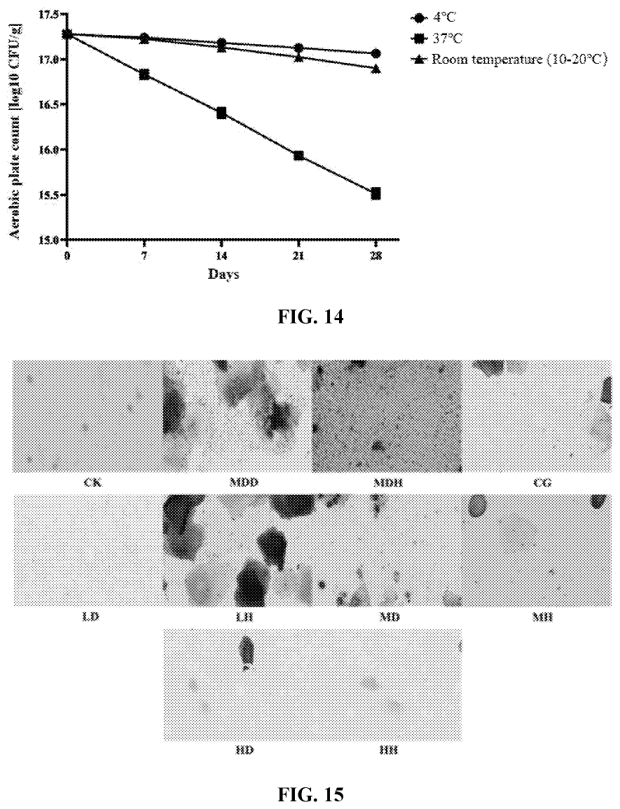

Using commercial kits, the mouse vaginal and uterine tissues are observed, and the vaginal tissues are subjected to HE and PAS staining; the levels of IL-1β, IL-6, and TNF-α in mouse serum are measured; the relative abundances of mouse intestinal bacteria, vaginal bacteria, and fungi are determined.
4) Experimental results: On the 4th day after administration, the vaginal lavage fluid is microscopically examined. The results are shown in FIG. 15, compared with the CK group and CKC group, a large number of Candida albicans are found in the MDD group and MDH group. Compared with the MDH group of the MDD group, the number of colonies in each administration group decreased, which is negatively correlated with the dose of administration; there is no significant difference between vaginal administration and combined administration.

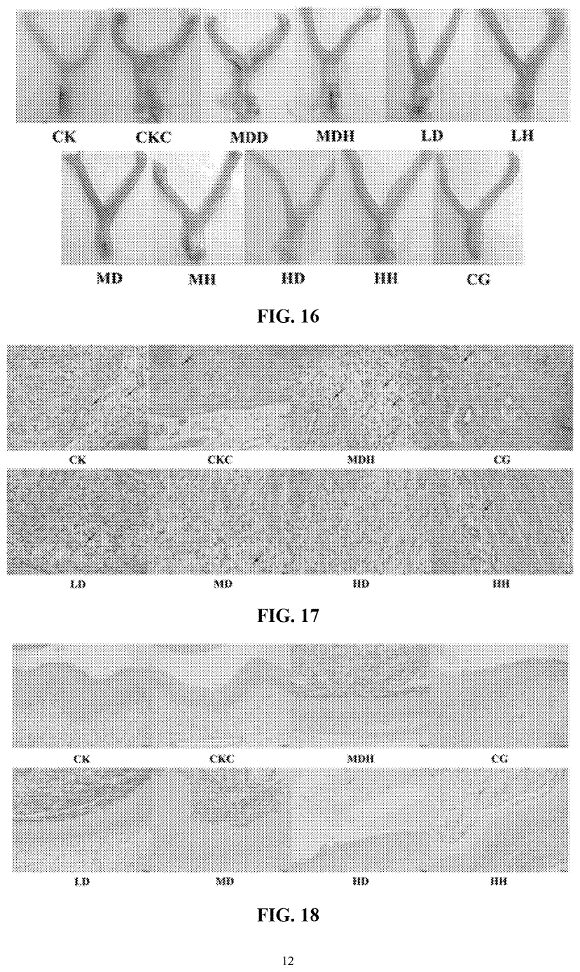

As shown in FIG. 16 that the tissue of the model group shows obvious pathological features such as redness, ulcer, and congestion, the size of the vaginal tissue in the administration group is small, and the redness and swelling are significantly improved, among which the high-dose group has the most obvious effect. In the high-dose group, there is no significant difference between vaginal administration and combined administration (vaginal administration, intragastric administration).

The representative pathological pictures are shown in FIG. 17 and FIG. 18. In the study of the mouse model of vaginitis, HE staining and PAS staining are significant. As a common staining method, HE staining can clearly present the cell structure and morphology of mouse vaginal tissue, such as the number and arrangement of epithelial cell layers, the distribution of blood vessels, connective tissue, and immune cells in the lamina propria, and help to understand the effect of vaginitis on tissue morphology. It can also be used to identify the type and number of inflammatory cells, such as neutrophils and lymphocytes, judge the severity of inflammation according to their aggregation and distribution, evaluate the modeling effect, and find pathological changes such as epithelial cell degeneration and necrosis, lamina propria edema, congestion and fibrous tissue hyperplasia, and help to explore the pathological mechanism of vaginitis.

PAS staining is used to detect fungi based on their specific staining reaction to polysaccharides. The fungal cell wall is rich in polysaccharide components, such as chitin, cellulose, etc. Periodic acid oxidizes the glycol groups in the polysaccharides of the fungal cell wall to aldehydes. The aldehydes then combine with the colorless basic fuchsin in Schiff's reagent to form a purple-red or red complex, thereby allowing the fungi to display a distinct color under the microscope for easy identification.

After careful microscopic observation of the tissue of the submitted sample, it is found that:

In the CK group and CKC group, the vaginal tissue structure is normal, and microabscesses could be seen in the mucosal epithelium, this is a common normal change in diestrus, mainly neutrophils, and no other obvious pathological changes are observed; a small amount of inflammatory cells is infiltrated in the lamina propria of mucosa in the CK group; some mice in CKC group are in proestrus, with mucous cells and positive PAS staining.

In the MDD group and the MDH group, fungi with positive PAS staining (red) are seen in the vagina, which should be Candida albicans according to the experiment. Microabscesses can be seen in the mucosal epithelium, which is a common normal change during diestrus, mainly neutrophils. There are more inflammatory cells in the lamina propria of the mucosa, with occasional edema.

In the CG group, LD group, LH group, MD group, MH group, HD group, and HH group, PAS staining positive (red) fungi could be seen in the vagina, which should be Candida albicans according to the experiment. Microabscesses can be seen in the mucosal epithelium, which is a common normal change during the diestrus, mainly neutrophils. A small number of inflammatory cells is infiltrated into the lamina propria.

The number of inflammatory cells (neutrophils, lymphocytes, macrophages, etc.) directly reflects the degree of inflammatory activity, and can judge the acute and chronic inflammation and the development process; the abnormal increase or decrease of the number of squamous epithelial cells indicate excessive or damaged epithelial cell proliferation, which is helpful to judge the degree of epithelial tissue damage. After administration, the number of inflammatory cells decreased, and the number of squamous epithelial cells returned to normal, which in most cases represented an effective improvement.

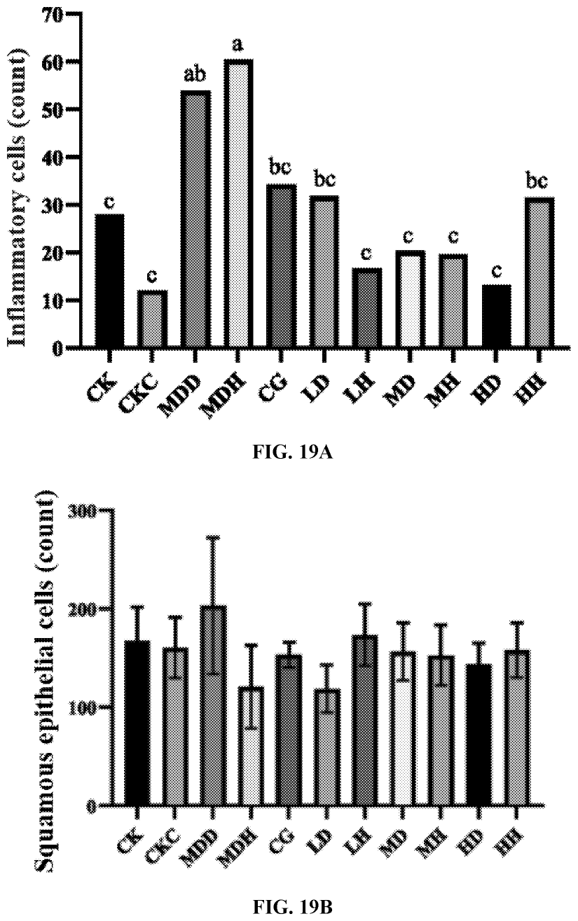

As shown in Table 3 and FIG. 19, the number of inflammatory cells in the lamina propria of each group (FIG. 19A) and the number of squamous epithelial cells (FIG. 19B) are enumerated. The results show that compared with the blank group, the number of inflammatory cells increases in the model group; the number of inflammatory cells decreases in all treatment groups, with the most significant reduction observed in the HD group (P<0.05). No significant difference is found in the number of squamous epithelial cells among the groups (P>0.05).

TABLE 3
Histopathological examination record form

| | HE | | | | | | |
|---|---|---|---|---|---|---|---|
| | Inflammatory cells | | | Squamous epithelial | | | |
| | (count) | | | cells (count) | | | |
| | Field | Field | Field | Field | Field | Field | |
| Group | 1 | 2 | 3 | 1 | 2 | 3 | Remarks |
| CK | 54 | 27 | 48 | 203 | 145 | 117 | Small abscesses in the epithelium |
| | 14 | 10 | 15 | 194 | 192 | 157 | Small abscesses in the epithelium |
| CKC | 11 | 10 | 16 | 111 | 196 | 154 | Small abscesses in the epithelium |
| | 11 | 14 | 11 | 190 | 165 | 149 | None |
| MDD | 20 | 70 | 35 | 167 | 217 | 117 | Numerous fungi and bacteria are |
| | | | | | | | visible in the vagina; small |
| | | | | | | | abscesses are in the epithelium |
| | 54 | 63 | 82 | 294 | 270 | 155 | Small abscesses in the epithelium; |
| | | | | | | | inflammatory cells are in the vagina |
| MDH | 136 | 117 | 15 | 96 | 98 | 81 | Numerous fungi and bacteria are |
| | | | | | | | visible in the vagina; small |
| | | | | | | | abscesses are in the epithelium; and |
| | | | | | | | edema in the lamina propria |
| | 16 | 43 | 35 | 122 | 198 | 130 | Numerous fungi and bacteria are |
| | | | | | | | visible in the vagina; small |
| | | | | | | | abscesses are in the epithelium |
| CG | 72 | 74 | 20 | 137 | 153 | 143 | Multiple small abscesses in the |
| | | | | | | | epithelium; numerous fungi and |
| | | | | | | | bacteria are visible in the vagina |
| | 16 | 15 | 9 | 170 | 151 | 167 | Small abscesses in the epithelium |
| LD | 15 | 5 | 7 | 141 | 157 | 101 | None |
| | 66 | 44 | 54 | 98 | 108 | 110 | Small abscesses in the epithelium; |
| | | | | | | | numerous fungi and bacteria are |
| | | | | | | | visible in the vagina, |
| LH | 29 | 18 | 11 | 175 | 168 | 231 | Small abscesses in the epithelium |
| | 17 | 13 | 12 | 137 | 172 | 160 | Small abscesses in the epithelium; |
| | | | | | | | a small number of fungi and bacteria |
| | | | | | | | are visible in the vagina |
| MD | 7 | 6 | 15 | 135 | 154 | 112 | Small abscesses in the epithelium; |
| | | | | | | | a small number of fungi and bacteria |
| | | | | | | | are visible in the vagina |
| | 90 | 59 | 8 | 191 | 177 | 171 | Small abscesses in the epithelium; |
| | | | | | | | a small number of fungi and bacteria |
| | | | | | | | are visible in the vagina |
| MH | 19 | 18 | 53 | 172 | 181 | 170 | Small abscesses in the epithelium; |
| | | | | | | | a small number of fungi and bacteria |
| | | | | | | | are visible in the vagina |
| | 6 | 10 | 12 | 123 | 167 | 106 | None |
| HD | 17 | 20 | 23 | 156 | 144 | 145 | A small number of fungi and bacteria |
| | | | | | | | are visible in the vagina |
| | 7 | 6 | 6 | 112 | 135 | 175 | Small abscesses in the epithelium; |
| | | | | | | | a small number of fungi and bacteria |
| | | | | | | | are visible in the vagina |
| HH | 34 | 47 | 68 | 150 | 120 | 174 | Small abscesses in the epithelium; |
| | | | | | | | a small number of fungi and bacteria |
| | | | | | | | are visible in the vagina |
| | 21 | 14 | 5 | 202 | 146 | 157 | None |

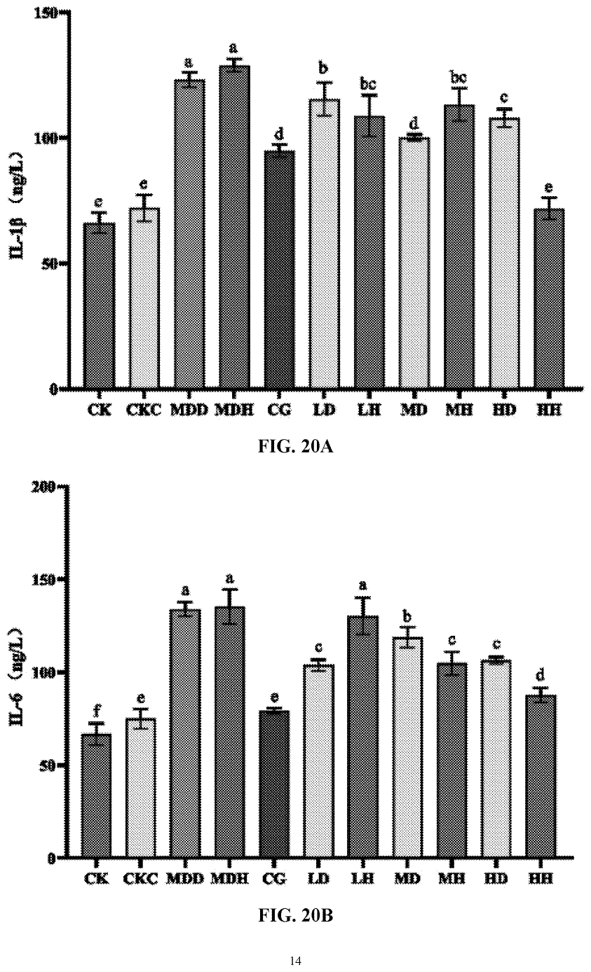

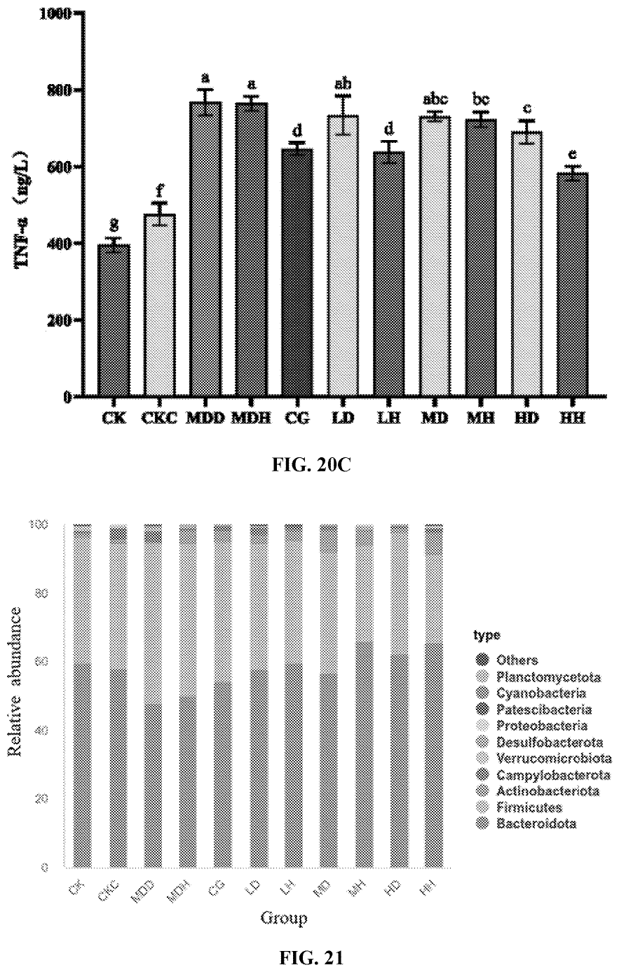

As shown in FIG. 20A, in the comparison of IL-1β levels, the serum IL-1β content in the MDD group and MDH group is significantly higher (P<0.01) than that in the treatment groups and the blank group. After treatment, as the dosage of the microecological preparation increases, the decrease in IL-1β shows an increasing trend. There is no significant difference (P>0.05) between the LD group, LH group, and MH group, as well as among the LH group, MH group, and HD group, indicating that, to a certain extent, the dosage has a minor effect on IL-1β. However, beyond a certain dosage, the effect became extremely significant (P<0.01); in combined administration and vaginal administration, only the high-dose combined administration group shows a better effect; the ability of commercially available lactic acid bacteria preparations to reduce IL-1β is higher than that of the low and medium dose groups, and lower than that of high-dose group; the level of IL-1β in the HH group is not significantly different from that in the control group (P>0.05), and the ability to reduce the level of IL-1β is excellent.

As shown in FIG. 20B, in the comparison of IL-6 levels, the serum IL-6 content in the MDD group and MDH group is significantly higher (P<0.01) than that in the blank group. After treatment, as the dosage of the microecological preparation increases, the concentration of IL-6 initially increases and then decreases, with the low-dose and high-dose groups showing better effects; there is no significant difference (P>0.05) between the LD group, MH group, and HD group, and among these three groups, indicating that, to a certain extent, the dosage has a minor effect on IL-6. However, beyond a certain dosage, the effect became significant (P<0.05); in combined administration and vaginal administration, the medium- and high-dose combined administration groups shows better effects; the commercially available lactic acid bacteria preparation is more effective in reducing IL-6 than the other treatment groups and shows no significant difference (P>0.05) compared to the control group; the HH group has a good ability to reduce the IL-6 level.

As shown in FIG. 20C, in the comparison of TNF-α levels, the serum TNF-α levels in the MDD group and the MDH group are significantly higher than that in the blank group (P<0.01); after treatment, as the dose of the microecological preparation increases, TNF-α shows a downward trend; there is no significant difference among the MDD, MDH, LD, and MD groups (P>0.05), indicating that low and medium doses of vaginal administration have almost no effect on TNF-α, while a significant effect is observed after reaching the high-dose group (P<0.05); in both combined administration and vaginal administration, all doses of combined administration are superior to vaginal administration; the commercially available lactic acid bacteria preparation shows a similar ability to reduce TNF-α as the LH group, but is inferior to the HH group; the HH group has a good ability to reduce the TNF-α levels.

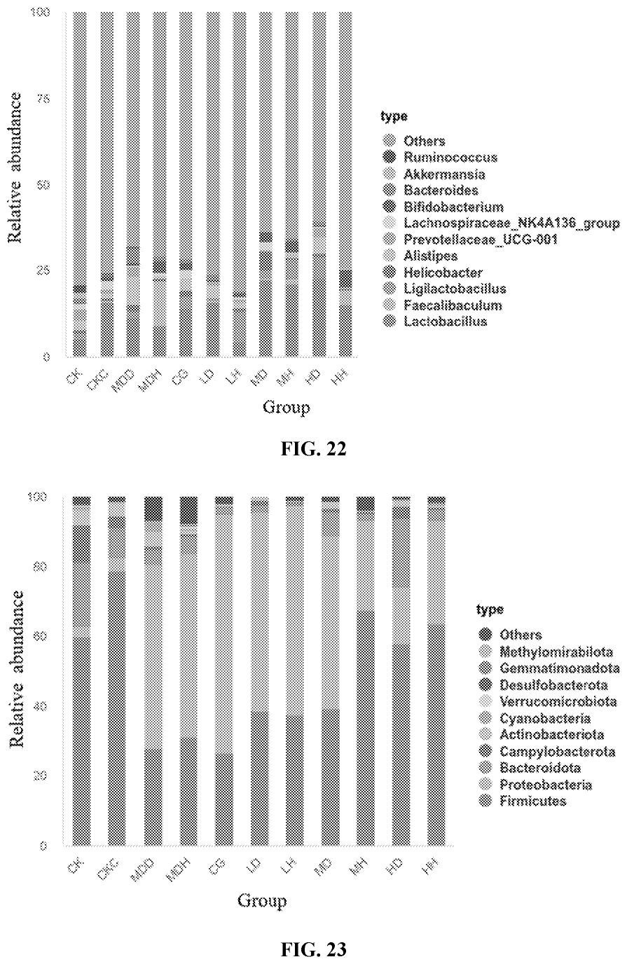

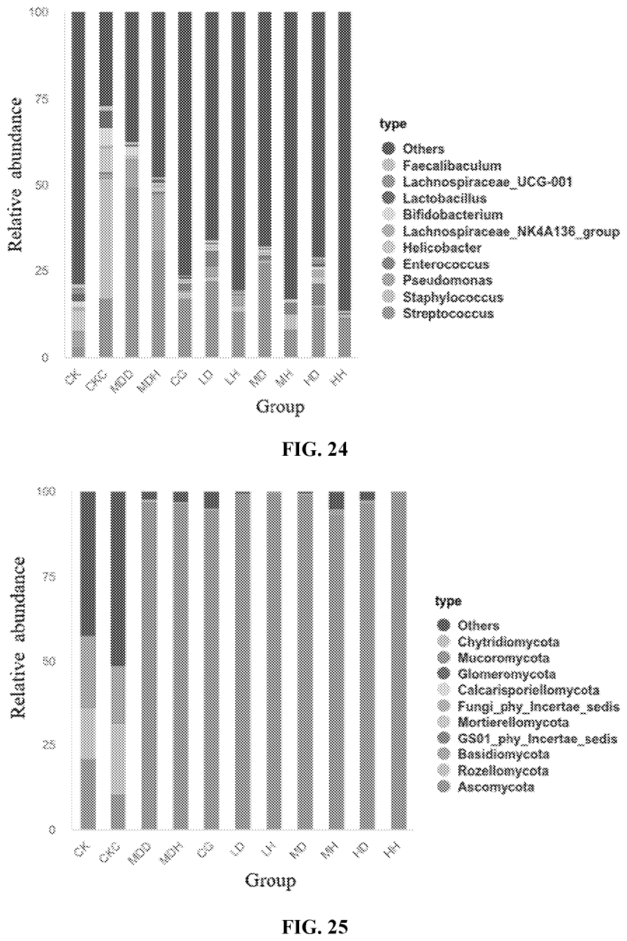

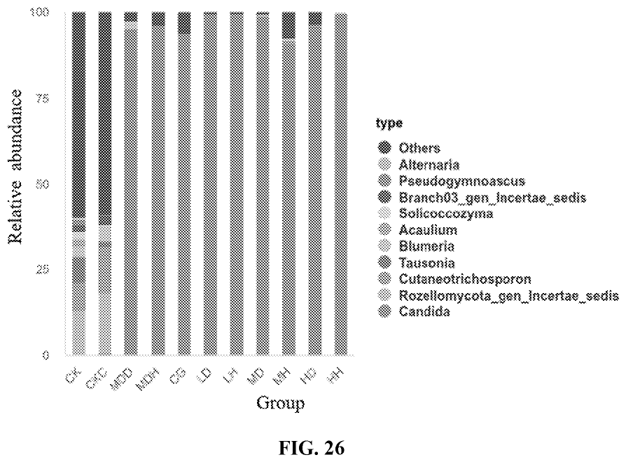

Through 16S rRNA gene sequencing analysis, as shown in FIG. 21, FIG. 22, FIG. 23, FIG. 24, FIG. 25, and FIG. 26, as the dosage increases, the relative abundance of Bacteroidetes in the mouse intestine increases, while that of Firmicutes decreases; in the HD group, the relative abundance of beneficial genera such as Lactobacillus and Ligilactobacillus accounts for nearly 30.6%, which is significantly higher than that in the model group (P<0.05). At the same time, the abundance of Firmicutes in the mouse vagina increases, while the abundance of Proteobacteria shows a trend of first increasing and then decreasing, the relative abundance of Proteobacteria in the HD and HH groups is lower than that in the other treatment groups, and the relative abundance of Streptococcus decreases significantly. In the mouse vagina, the relative abundance of Ascomycota decreases in the HD group, while the relative abundance of Candida decreases in the CG, MH, and HD groups. Both the HD and HH groups improve the species richness and diversity of the intestinal and vaginal microbiota to some extent, and the community structure gradually converges toward that of the CK group.

The above description is not a restriction on the present disclosure, and the present disclosure is not limited to the above embodiments. The changes, modifications, additions, or replacements made by ordinary technical personnel in the technical field within the substantive scope of the present disclosure should also belong to the protection scope of the present disclosure.

What is claimed is:

1. A probiotic with efficacy in improving vaginitis, wherein the probiotics comprise at least one of a Lactiplantibacillus plantarum 22-9 strain with a deposit number CGMCC No. 33867, Lactiplantibacillus plantarum XA-9 strain with a deposit number CGMCC No. 33868, and Lactiplantibacillus pentosus B-1-35 strain with a deposit number CGMCC No. 33870.

2. A probiotic compound microecological preparation with efficacy in improving vaginitis, the probiotics comprise at least one of Lactiplantibacillus plantarum 22-9 strain with the deposit number CGMCC No. 33867, Lactiplantibacillus plantarum XA-9 strain with the deposit number CGMCC No. 33868, and Lactiplantibacillus pentosus B-1-35 strain with the deposit number CGMCC No. 33870, wherein active ingredients of the probiotic compound microecological preparation comprise probiotic bacteria.

3. The probiotic compound microecological preparation with efficacy in improving vaginitis according to claim 2, wherein a number of viable bacteria in the probiotic compound microecological preparation is not less than 1× 10⁷ CFU/mL.

4. The probiotic compound microecological preparation with efficacy in improving vaginitis according to claim 2, wherein a concentration of Lactiplantibacillus plantarum 22-9 bacteria solution, Lactiplantibacillus plantarum XA-9 bacteria solution and Lactiplantibacillus pentosus B-1-35 bacteria solution is the same and a mixed volume ratio is 2:1:3.

5. A method for preparing the probiotic compound microecological preparation with efficacy in improving vaginitis according to claim 2, wherein the method comprising the following steps:
(1) inoculating Lactiplantibacillus plantarum 22-9, Lactiplantibacillus plantarum XA-9, and Lactiplantibacillus pentosus B-1-35 into a fermentation medium at a volume ratio of 1-5%, and culturing at 35-40° C. for 15-20 h to obtain a lactic acid bacterial culture, mixing the lactic acid bacterial culture of Lactiplantibacillus plantarum 22-9, Lactiplantibacillus plantarum XA-9, and Lactiplantibacillus pentosus B-1-35 at a volume ratio of (1-3):(1-3):(1-3) to obtain a mixed starter culture, inoculating mixed starter culture into the fermentation medium at a volume ratio of 2%, and culturing at 37° C. for 18 h to obtain a mixed culture of lactic acid bacteria;
(2) centrifuging the lactic acid bacterial culture obtained in step (1) to collect a lactic acid bacteria pellet, and adjusting a cell concentration to 1×10⁷-1×10¹⁰ CFU/mL using Phosphate Buffered Saline (PBS), adding lyoprotectant of the same volume as sterile PBS, mixing and placing in a freezer at −80° C. overnight for pre-freezing, freeze-drying the pre-frozen sample to obtain a probiotic compound microecological preparation.

6. The method for preparing the probiotic compound microecological preparation with efficacy in improving vaginitis according to claim 5, wherein the lactic acid bacterial culture of Lactiplantibacillus plantarum strain 22-9, Lactiplantibacillus plantarum strain XA-9, and Lactiplantibacillus pentosus strain B-1-35 are mixed in a volume ratio of 2:1:3 to obtain a mixed starter culture, and the bacterial concentration of each lactic acid bacterial culture is the same.

7. The method for preparing the probiotic compound microecological preparation with efficacy in improving vaginitis according to claim 5, wherein the fermentation medium described in step (1) is prepared by dissolving an MRS solid medium with 1 L of distilled water and sterilizing the MRS solid medium at 121° C. for 15 min, composition of the MRS solid medium comprises: 10 g of peptone, 10 g of beef extract, 5 g of yeast extract, 2 g of diammonium citrate, 5 g of sodium acetate, 20 g of glucose, 1 mL of Tween 80, 0.5 g of magnesium sulfate, 0.25 g of manganese sulfate, and 15 g of agar powder.

8. The method for preparing the probiotic compound microecological preparation with efficacy in improving vaginitis according to claim 5, wherein a formula of lyoprotectant described in step (2) is 118.2 g/L of trehalose, 17.1 g/L of L-cysteine, 10.3 g/L of sorbitol, 1.7 g/L of sodium acetate, and 120 g/L of skim milk powder.

9. An application of the probiotics according to claim 1 in a preparation of Gardnerella vaginalis and/or Candida albicans inhibitors.

10. An application of the probiotic compound microecological preparation according to claim 2 in a treatment of vaginitis symptom improvement or therapeutic drugs.
